(19) United States
(12) Patent Application Publication
(10) Pub. No.: US 2009/0082867 A1
(43) Pub. Date: Mar. 26, 2009

(54) INTERVERTEBRAL DISC PROSTHESIS FOR UNIVERSAL APPLICATION

(76) Inventors: Cesar Sebastian Bueno, Malaga (ES); Ignacio Abad Rico, Malaga (ES); Francisco Manuel Garcia Vacas, Malaga (ES); Francisco Ezquerro Juanco, Malaga (ES); Antonio Simon Mata, Malaga (ES)

Correspondence Address: STITES & HARBISON PLLC, 1199 NORTH FAIRFAX STREET, SUITE 900, ALEXANDRIA, VA, 22314, US

(21) Appl. No.: 11/908,358

(22) PCT Filed: Sep. 8, 2004
(86) PCT No.: PCT/ES2004/000395

§ 371 Date: Nov. 17, 2008

(51) Int. Cl. A61F 2/44

(52) U.S. Cl. 623/17.16; 623/17.11

(57) ABSTRACT

An Invertebral Disc Model Prosthesis, of the type denominated of double articulation, to substitute the function and the movement of intervertebral discs, for universal application due to its principal characteristic of being able to be used as a constrained, semi-constrained or non-constrained prosthesis, specially indicated for treatment of pathological degenerative of the intervertebral discs, discal hernias, by anterior approach, transition syndromes of supra-adjacent disc, chronic lumbagos resistant to conserver treatment, chronic adjacent vertebral instability, made from materials of proved biocompatibility, endowed with a low profile that makes it optimum for its implant in the human being and which consists of three pieces, two plates, upper and lower, and am intermediate piece , which serves for the substitution of the discs of the lumbar and cervical column, capable of being placed by the anterior or lateral approach.

### 1. OBJECTIVE OF THE INVENTION

The objective of this invention is a new model of Modular Disc Prosthesis with which the functions of the natural human intervertebral disc will try to be reproduced, achieving a totally controlled physiological movement, without overloading small backward articulations and with the possibility of adapting the implant to each particular case according to the degree of discal deterioration that each patient experiences at that moment. To obtain these two objectives a modular prosthesis has been developed, which can be used in three versions, constrained, semi-constrained and non-constrained, to be adapted to each patient with an anatomical-functional design that enables its implantation and a primary solid and firm stability because of its covering and the form of anchoring to the bone, which constitutes a low friction implant stability due to the materials used on its sliding surfaces; with highly wear resistant materials which enable implantation by anterior approach, as all the existing ones, and also by lateral approach.

The objective of this invention is achieved using a disc prosthesis as claimed in the previous claims.

### 2. BACKGROUND

To date, many attempts have been made to develop a substitute for the human intervertebral disc, which can deteriorate due to early wear, arthrosis and repeated traumatisms. The first solution possible to resolve this problem, widely used in the past, was the total annulment of the disc as a mobile element, by the substitution thereof with bone implants, which provoke the total fusion or welding and the joining of the two adjacent vertebrae supported by vertebral plates and screws (called vertebral staples) which were introduced through the vertebral peduncles. But the problem was not resolved definitively with the arthrodesis or fusion, that is to say with the annulment of mobility on a vertebral segment, formed by two adjacent vertebrae joined to each other by an intervertebral disc, above all in young patients. On these patients, upon eliminating one or various consecutive discs the initial problem was momentarily resolved, but all work and bio-mechanical requirements is transferred to the free disc immediately above or below which corresponded to the disc or fused discs. This causes that after a few years the free overlaid or underlaid discs start to degenerate gradually until they reach complete destruction, producing what is known as Adjacent Disc Syndrome or Transition Space Syndrome.

To prevent that this Primary Discal Degenerative Pathology be resolved with a blocking method such as Fusion, that can subsequently generate a new Progressive ascending or descending Degenerative Pathology, the development of the Disc Prosthesis was thought of.

Within this Degenerative Pathology of invertebral Discs, we can find two types of degenerated discs that have completely different bio-mechanical behaviors:
a) Degenerated Hypermobile Disc, more common in younger persons, particularly middle aged women, and which is characterized by having a movement range in the three axes of space, much superior to normal, exceeding the physiological limits permitted above all in the flexo-extension and in axial rotations, which normally results in painful lumbago and intermittent pseudo-sciatic pains and symptoms. This situation corresponds in general, with the first stages of Kirkaldi and Wyllis Vertebral Instability Syndrome.
b) Degenerated Hypomobile Disc, in which the mobility of the segment is much reduced, below the physiological demands, which are made above all, in older persons and which correspond in general, with more advanced states of the Vertebral Instability Syndrome. In these cases, the intervertebral space is more reduced and calcifications, osteophytes and reparative and degenerative phenomena appear in the small articulations with hypertrophies of the same and a rigidity in all the elastic contention elements such as the ligaments and articulating capsules of the segment.

Among the two types of hyper and hypo-mobile discs can be found cases of Degenerative Normo-mobile Discs in which the range of movement is within normal limits and therefore, the treatment given to each case, has to be different.

It is important that all the foregoing must be taken into account at the time of selecting the type of prosthesis to be used, depending always on the type of Degenerative Disc damage suffered by the patient, duly making exhaustive studies of each case in particular both clinically and radiologically, with all kinds of image studies (radiographs dynamic, computerized axial tomographs, magnetic resonance, etc.) to be able to define which type of Degenerative Disc Damage is shown by the patient and also to be able to select the type of prosthesis needed.

### 3. STATE OF THE ART

At present, there are more than 127 different models of Disc prostheses registered, of which almost 98% have never been used in human beings; of those that have been used, many have failed due to design errors, failures in stability, or for being made of unsuitable materials. Nevertheless, today there are in the market, some models which have been used in a wide variety of patients and for over 17 years have been inserted with total acceptance on the part of international bodies for the control of implants and prostheses in the human body.

Although it is true that the latest designs have improved in a general way in their design and quality of materials, it is no less certain that they still suffer from significant defects that justify the need to perfect the configuration of these disc prostheses to make them more like the actual function and anatomy that the human intervertebral disc requires.

The first implantation of an artificial disc was done by Fernström in 1964 (Steel ball bearings). This first disc consisted of a metal ball bearing implanted directly between the vertebral bodies and its use was discarded due to the tendency it showed for the subsidence of the implant in the vertebral bodies as well as problems of hypermobility of the vertebral segment. From these first implants until the present, numerous models and prototypes of intervertebral disc prostheses have been carried out, many of the designs having been patented and several of them used surgically.

All these designs follow the main tendencies: a) to simulate the elastic or visco-elastic properties of the disc, in those that govern the capacity to dissipate energy or the absorption of axial loads or b) simulate the natural physiological mobility characteristics of the disc.

a) The prostheses, which try to reproduce the elastic or visco-elastic properties of the disc are characterized by being connected to both vertebral bodies through mechanical elements with some suitable elastic properties, in such a way that the rigidity they confer to the unit in the different axes of movement shall be similar to those corresponding to a healthy intervertebral disc. In some cases, these designs add elements with visco-elastic properties aimed at also simulating the visco-elastic properties of the healthy disc. The materials used for obtaining these objectives are mainly silicones, polymers, rubber or even metal springs. There are various published or patented designs of this type of prosthesis, although very few have been implanted.

Among said prostheses we can find the one designed by Fassio, discarded for problems of subsidence of the implant; the Acroflex which, after an evolution of the original design seems to have also been discarded due to the appearance of defects in the material used, polyephiline, after 1-2 years from its implantation; and lastly, the Bryan cervical prosthesis. It has practically gone out of use today, because what matters the most, is to reproduce the physiological movement of the disc.
b) For its part, the prostheses designed with the criteria of simulating the mobility properties of the intervertebral disc are the most used at present, in the surgery of discal pathologies, due to their better clinical behavior. The prostheses emulate the concepts used satisfactorily in the design of prostheses of other articulations such as the knee and the hip. Its basic functioning consists of enabling the relative movement between the vertebral bodies that connect through sliding between the surfaces of the different components. Within this type of prosthesis two tendencies can be distinguished: on one hand there is the constrained, and on the other the non-constrained design:
b-1) The constrained prostheses, among which are presently found the so-called Pro-Disc, Maverick, Prestige (Bristol) or Flexi-Core, enabling relative rotation between the vertebrae, but done in such a way that the axis with respect to which rotation is carried out, is fixed by the mechanical design of the prosthesis. In these types of prostheses the sliding surfaces are spherical heads, for which reason the axis with respect to which rotation is made in each anatomic plane is a fixed axis that passes through the geometric center of the spheres. The location of this axis is determined by the position of the spherical surfaces inside the intervertebral space as well as by its radius, in such a way that no matter how big this radius may be, the rotation axis will be found further away from the intervertebral space. In turn, the position of the axis of rotation affects the magnitude of transversal displacement associated with the rotation, in such a way that in general, the lower its position may be, the greater will be the magnitude of the same. (See FIG. 1).

Among the constrained designs existing, there is a kind of constructive solution based on using sliding surfaces (S1, S2) of large radius (R) located in the center of the intervertebral space (FIG. 1). With this configuration, the center of rotation (C), both for flexo-extension and lateral extension movements, would be located in the central zone of the lower vertebral body. This position does not correspond with the results of the studies published on the kinematics of the healthy lumbar column, which place this center of rotation in different positions along the movement, but always in regard to the intervertebral space. As a consequence, this type of design will produce a forced movement of the interapophysary articulations, which would lead to a rise in mechanical stresses supported by the same. This is the case of the Pro-Disc Prosthesis.

The other solution used in constrained designs (Maverick and Flexi-Core) is to use sliding surfaces (S1, S2) of smaller size with which the axis of rotation (C) is moved closer to the intervertebral space (FIG. 2) on the basis of having to use a more resistant material, since UHMWPE (Ultra High Molecular Weight Polyethylene), used in the former solutions, cannot resist the high stresses generated in these designs. In the lumbar prosthesis, the location of the sliding surfaces of these designs tend to be in the back third of the intervertebral space. With this location, the center of rotation of the flexo-extension movements is also situated in this back zone, simulating better the extension movements of the segment, but drifting away from the physiological behavior in the flexing movements.

The fundamental problem that this type of prostheses have is that they do not enable a physiological displacement in the anterior-posterior or transversal sense and this constitutes an important defect, that assumes a bigger overload of the small articulations and as they are provided with only one sliding surface, the degree of friction and wear is much greater and more advanced.

In a healthy vertebral segment, the relative movement between the vertebrae is determined by the interaction between the intervertebral disc, the interapophysary articulations, and the ligaments. The action of each one of these structures determines the location of the axis of rotation of this movement, which is variable in the length of the same. Therefore, none of the former constrained designs enable the restoration of physiological movement of the movement segment.

b-2) Non-constrained designs enable both independent and rotational displacements in the three anatomical axes. With this type of design, it is attained that the axis, with respect to which rotations in each one of the anatomical planes are produced, is not fixed by the prosthesis itself, but by the combined action of the prosthesis, ligaments, rear articulations and muscles which determine the said axis at every moment.

Among the existing unconstrained designs, there are at present prostheses such as those of Charité and Moby-Disc. Both designs have two pairs of sliding surfaces for the transmission of movement of one vertebra to another. In the case of Charité each one of the two pairs of surfaces are spherical, while in the Moby-Disc both upper surfaces are spherical and the lower ones are flat.

The Charité was developed at the beginning of the 80's, a great number of them having been implanted, and numerous studies having been published on the same. This prosthesis consists of three components: a central nucleus of polyethylene, and two metal pieces which contain it. The upper and lower surfaces of the nucleus are convex and each one of them makes contact with the corresponding concave surfaces of each of the metal pieces which enclose the nucleus, these pieces being anchored to the vertebral bodies. With this design each mechanism enables flexo-extension and lateral flexion of 2 degrees of free movement and therefore, the prosthesis does not stress the position of the center of rotation.

The Moby-Disc maintains practically the same concept, but both in turn can produce sliding shear in excess, which, on not being able to be absorbed by the prosthesis impact on the rear articulations, generate a facetary syndrome that results in great pain.

This type of non-constrained design enables various possibilities of relative movement between vertebrae, which supports the advantage of allowing a more physiological movement to the vertebral segment. Nevertheless, this multiplicity of possible movements may also constitute an inconvenience as there may be in existence excessive translation movements that oblige the transmission of significant shear forces between vertebrae, which on not being able to be absorbed by the prosthesis, must be transmitted through the rear articulations. This type of problem means that the decision between a constrained and a non-constrained prosthesis is not clear, for which reason both types of prosthesis co-exist in the market at present, leaving the surgeon to make the final decision on what type of design to use.

### 4. BRIEF DESCRIPTION OF THE DRAWINGS

In order to facilitate a better understanding of this descriptive memorandum and forming an integral part of the same, a series of Figures is attached in which by way of illustration and not limiting in any way, the following is represented:

FIG. 1 shows a set of two vertebrae with an intervertebral disc in two rotational positions, where the axis of rotation is in the intermediate zone to the lower vertebra.

FIG. 2 shows a set of two vertebrae with an invertebral disc in two rotational positions, where the axis of rotation is in the upper zone of the lower vertebra.

FIG. 3 shows the intervertebral disc prosthesis of the invention expanded in perspective and in elevation.

FIG. 4 shows an arrow-shaped section of the invention prosthesis in its non-constrained configuration.

FIG. 5 shows a perspective of the invention prosthesis in which the left rear quarter has been cut out to give a better view.

FIG. 6 shows a perspective and a plane view of the invention prosthesis in its configuration for entry by the side path.

FIGS. 7a and 7b show arrow shaped sections of the invention disc prosthesis in its constrained and non-constrained configuration respectively.

FIGS. 8a and 8b show sections of the invention disc prosthesis that illustrate the maximum displacement permitted by said prosthesis in the forward and backward directions respectively.

FIG. 9 shows a mechanism equivalent to the constrained configuration of the invention disc prosthesis.

FIG. 10 shows the instantaneous axis of rotation of the invention prosthesis in the flexo-extension movements, position A being neutral, B a flexion of 12° with respect to A, and D and an extension of 9°with respect to A.

FIG. 11 shows the differences in the forward-backward displacement relative to the articulating faces in using a pair of spherical contact surfaces (left) or the invention prosthesis in its constrained configuration (right) for a flexion of 6°.

FIG. 12 represents two arrow-shaped sections of the invention prosthesis showing the positions adopted by the components of the semi-constrained prosthesis in the maximum range of flexion and extension respectively.

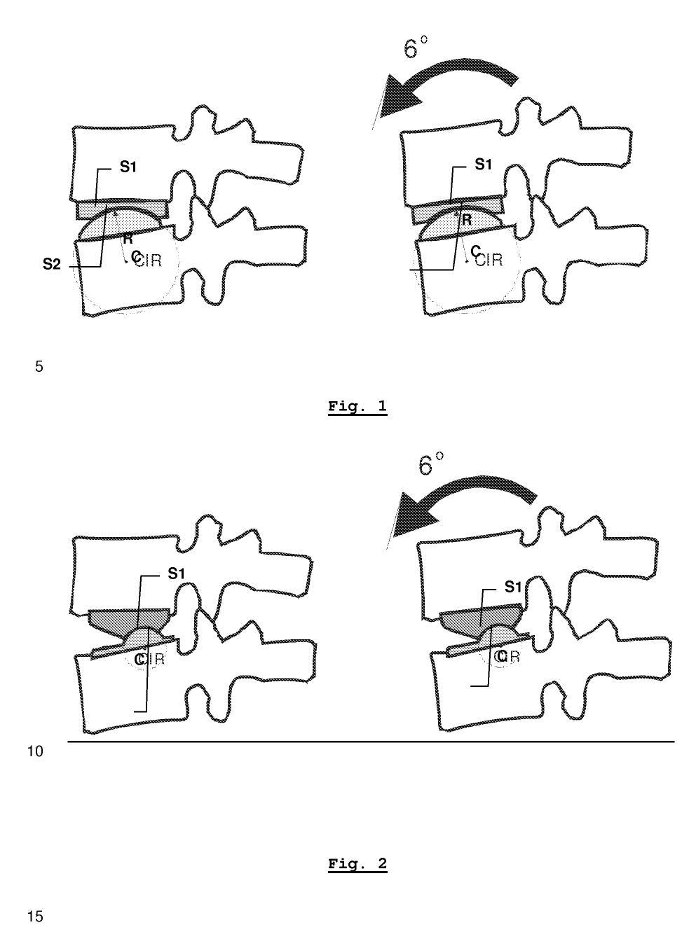

### 5. DETAILED DESCRIPTION OF THE INVENTION

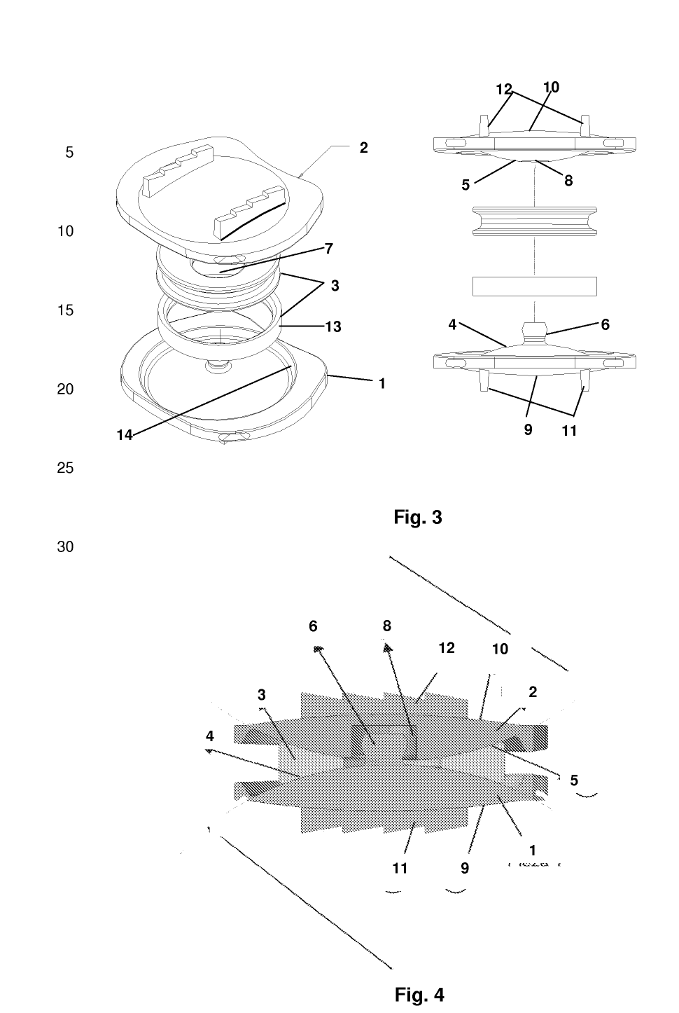

The invention intervertebral disc set prosthesis (FIGS. 3 and 4) consists of three different pieces:
a lower piece (1) which will be fixed to the lower vertebra,
an upper piece (2) anchored to the upper vertebra.

Both are made of an alloy of Chrome-Cobalt-Molybdenum covered on its outer surface with a tantalum plasma for better osteo-integration.

The lower (1) and upper pieces (2) are alike to each other, with the exception of the following: Both have lenticular shape in vertical section and a kidney in plane view. The two pieces (1, 2) show similar traces of spherical surfaces (4, 5, 9, 10) on their upper and lower faces but the spherical heads that conform to the lower surfaces (5) of the upper piece (2) and the upper (4) of the lower piece (1), that are the operative sliding surfaces of the articulation, having a radius of curvature of less than that of the upper spherical heads (10) of the upper piece (2) and lower face (9) of the lower piece (1). The relative positions of the elements are in accordance with the prosthesis in its operative position as represented in FIG. 4. The spherical operative surfaces (4, 5) end in contention rims (14) that act as a stop to the displacement of the intermediate piece (3). The spherical surfaces (9,10) opposite the operatives cover almost the whole of the upper faces of the upper piece (2) and the lower of the lower piece (1) that terminate in a flat edge.
an intermediate biconcave torroidal piece (3), made of Cross Link Ultra Heavy Molecular Polyethylene (CLUHMWPE) which articulates on the other two pieces. The upper and lower cavities of this intermediate piece (3) are spherical with substantially the same curvature as the operative surfaces (4, 5) of the lower (1) and upper pieces (2) with the object that these are in contact over the whole surface when the said pieces (1, 2, 3) are mounted in their operative position. This third piece of polyethylene is fitted with a titanium metal ring (13) around its circular outside edge, which confers on the same, a bigger resistance at the same time it serves to visualize its position by means of radiographies. The articulation between the pieces (1) and (3) as well as between pieces (2) and (3), is done by means of sliding between each pair of spherical surfaces (4) and (5).

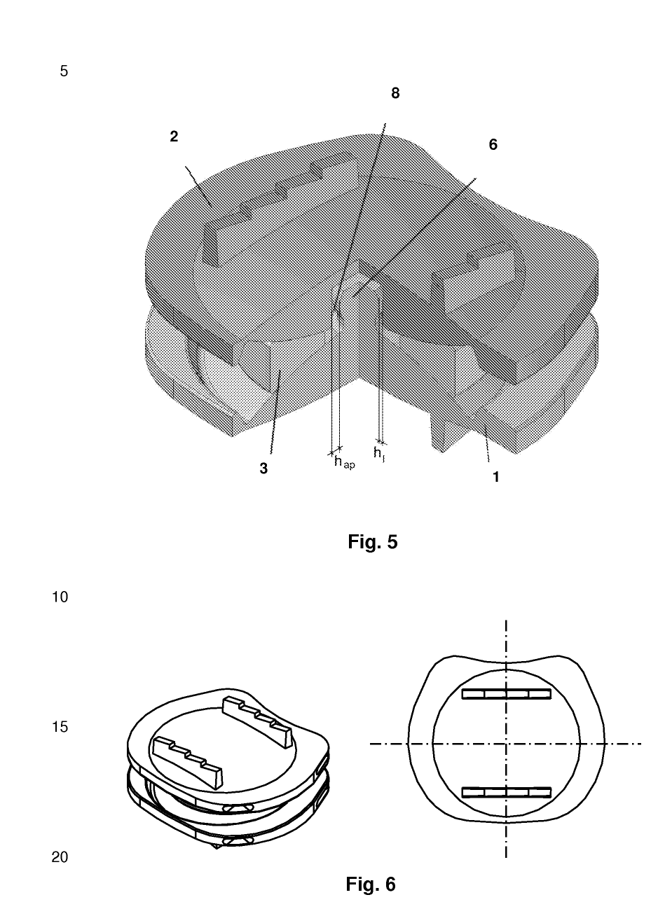

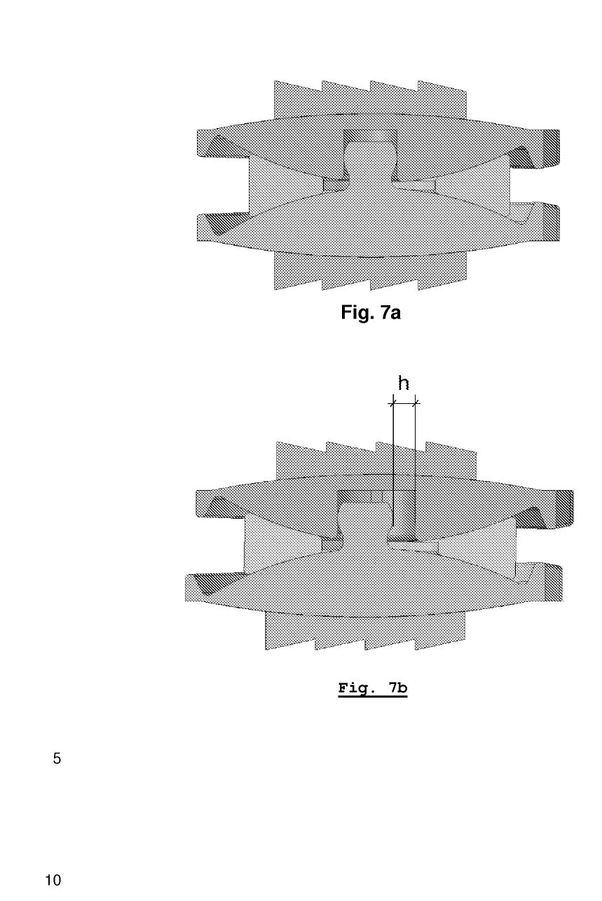

In addition, the lower piece (1) has a pivot, which radially divides from the top of the surface (4) and finishes in a sphere (6). This pivot passes through the intermediate piece (3) via the central orifice (7), which it has, and is lodged in the hole (8) inserted in the upper piece (2). Alternatively, the spherical head pivot (6) and the hole (8) can be displaced in the saggital plane with respect to a centered position in such a way that the range of mobility of the prosthesis in flexion may be more than in extension. The play between the pivot sphere (6) and the hole (8) depends on the difference between the transversal dimensions of this hole and the diameter of the sphere. In the design of the prosthesis of this invention, the following three situations are foreseen:
a) Constrained prosthesis: the hole (8) is cylindrical with a diameter substantially equal to that of the pivot sphere (6) (FIG. 7A). In this case, there is no play between both components, thus making a constrained design of the prosthesis.
b) Semi-constrained prosthesis: the transversal dimensions of the hole (8) are larger than the diameter of the pivot sphere (6) so there is a play between the two elements. This play can be characterized by a value h when the hole (8) is cylindrical, or between two values hap: play in the anterior-posterior direction, and hi: play in the middle-lateral direction (FIG. 5 and 7b); in this case the hole (8) is not cylindrical but its section is ellipsoidal. In this design situation the plays h do not exceed 1.5 mm.
c) Non-constrained prosthesis: in this case the play h (or hₐₚ, hₗ) is between 1.5 and 3 mm, which enables more displacement.

Fixing the pieces (1) and (2) to the vertebral plates is done through the outer surfaces of each of these pieces, that is to say, from the lower surface (9) for the lower piece (1) and from the upper surface (10) of the upper piece (2). These surfaces have a slightly convex shape with the object of molding itself better to the surface of the vertebral plates, and each one of them has two crests or spikes (11) (12) parallel to each other, that must be nailed to the vertebral plates. The design of present prosthesis contemplates two possible arrangements for these crests in function of the selected approach route:
1) Parallel to the anterior-posterior direction for implantation by anterior approach (FIG. 5).
2) Parallel to the lateral direction for implantation by lateral approach (FIG. 6).

### 6. FUNDAMENTAL ADVANTAGES OF THE PRESENT INVENTION DISC PROSTHESIS

The most important advantages of the invention prostheses is centered in the following aspects: 1°.—Bone anchorage to the vertebral plates; 2°.—Nodular character of the prosthesis 3°.—Wear and resistance of the material, 4°.—Way of entry and 5°.—Possibility of selecting the most suitable type of implant.

### 6.1.—Bone Anchorage

It is very important to obtain a fixing and stability of the prosthesis in an advanced approach, which would ensure initial stability, at least during the first three months, until total osteo-integration is achieved. Even with a good preparation of the vertebral bed, it is essential to have a tight contact of the outer surface of the prosthesis with the vertebral plates, which in the majority of cases, use to have a concave morphology more or less centered, which can leave the prosthesis with scant support and therefore, with weak stability being able to cause an advanced mobility of the same.

To avoid the foregoing problem, the invention prosthesis has an outside surface with a discreet convexity which enables a good seating on the natural concavity of the vertebral plates. Otherwise, this concave morphology of the vertebral plate obliges a milling adjustment of the same, which is translated into a weakening of the bone structure and seating base and as a result the possibility that subsidence of the Prosthesis in the vertebral plate will be produced. Because of this, the Invention Disc Prosthesis is fitted with slightly convex external seating surfaces (9, 10) in both vertebral plates.

Besides, in order that its adherence to the bone should be the firmest possible, from the first moment it is also necessary that the external surfaces (9, 10) in contact with both vertebral plates are covered with a material that is bio-compatible and easily osteo-integrating, such as occurs in the invention disc prosthesis, which is covered with a porous plasma of Tantalum. In addition, both external surfaces (9, 10) are fitted with two lines of metal crests with spikes (11, 12) that will attach solidly to the spongy material of the vertebrae bodies during the first weeks of the implantation.

### 6.2.—Modular Character if the Prosthesis

The invention prosthesis, which can be called modular or multifunctional, can behave as a constrained, non-constrained or semi-constrained prosthesis according to the needs of each case. It has a double sliding surface (each one of the surfaces 4 and 5 with the corresponding surfaces of the intermediate piece), with the possibility of displacement in the horizontal, both transversal and limited anterior-posterior sense and a third articulation metal-metal between the small sphere of the central pivot (6)and its housing (8) in the upper piece (2).

The characteristic of being able to act as a constrained, or semi-constrained prosthesis goes in function of the relative dimensions given between the sphere (6) of the lower piece (1) and the hole (8) of the upper piece (2).

Just as can be seen in FIG. 7a, if the dimensions of said sphere and hole substantially coincide, the prosthesis will act as a constrained prosthesis, that is to say, it will have only on degree of free movement in each one of the anatomical planes and therefore, the instantaneous axis of rotation will be forced by the design of the prosthesis. However, if these dimensions do not coincide there will be a play “h” (FIG. 7b) that will enable more than one degree of free movement in each anatomical plane. When said play “h” is inside a small range (not more than 1.5 mm.) we would be faced with a case of a semi-constrained prosthesis in which the relative movement will not be forced by the design of the prosthesis in such a way that it will be the combined action of ligaments, articulating faces and muscles that determine the location of the rotation axis. If said play “h” is between 1.5 and 3 mm, this enables a bigger displacement without exceeding the physiological limits and will be faced with the third possibility of a non-constrained prosthesis.

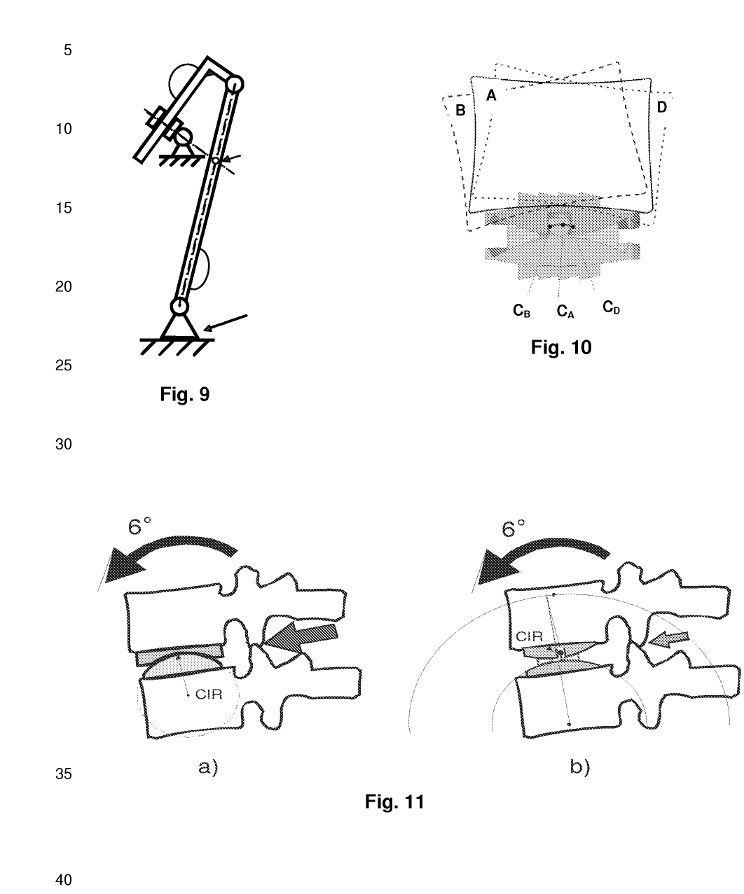

When the constrained configuration of the prosthesis is used (h=0), the relative movement, both in a saggital and frontal plane, between the two vertebrae connected by the same, can be simulated by means of the mechanism represented in FIG. 9, where a link represents the intermediate element and the other link the element anchored to the upper vertebra. When the lower vertebra is considered fixed, the movement of the upper vertebra in each one of the anatomical planes cited, consist of a rotation plus a translation. At any instant, this movement can be defined as an instantaneous rotation with respect to an axis: the instantaneous axis of rotation. The position of this axis in the present prosthesis is different for each position adopted by the upper vertebra. In FIG. 10 the successive positions can be seen, that are adopted by the axis of rotation (C_{A}, C_{B}, C_{D}) of the flexo-extension movements carried out in the plane of the figure; these positions have been represented by points, since for said movements, the axes are perpendicular to the plane of the figure. As can be evidenced, this axis is always on the central zone of the intervertebral space, displaced in sense (anterior) when the section is flexed and in the opposite sense (posterior) when it is extended.

This characteristic of movement is similar to that observed by White and Panjabi in a lumbar movement unit, for which reason it can be considered that the present prosthesis, in its constrained configuration enables the reproduction of a movement resembling more the physiological one than any other of the present existing constrained prostheses. In addition, the location of the axis of rotation in the central zone of the intervertebral space enables the minimizing of the relative transversal movement between the articulating faces (FIG. 11) with the consequent reduction of mechanical tensions supported by the same.

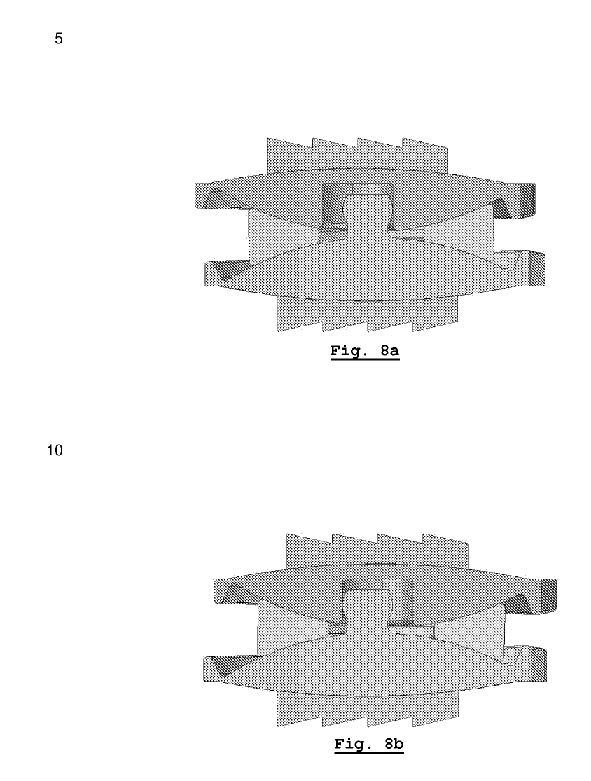

When the non-constrained configuration of the prosthesis (h≠0) is used, the relative movement between the vertebrae will have six degrees of freedom, with which the rotating movements enabled by the prosthesis will be independent of the movement of translation. Therefore, with this configuration, the instantaneous axis of rotation for the movements in different anatomical planes will not be defined by the relative position between the vertebrae, but as it happens in any design of non-constrained prosthesis, it will be defined by the actuation of different structures that connect both vertebrae. One problem associated with this type of design is the possible existence of excessive transversal displacement. In the present prosthesis the displacement will be always limited by the value of the play h (FIGS. 8a and 8b). In this figure can de seen respectively, the maximum anterior and posterior displacements allowed by the prosthesis. In addition, the value of the play ‘h’ can be dimensioned independently for the anterior-posterior direction and for the lateral direction (perpendicular to the anterior-posterior direction) making the hole (8) have for example an elliptical shape, for which reason the maximum displacements in both directions can be limited independently. Depending on the amplitude of the play “h” we would obtain a totally non-constrained or rather, a semi-constrained prosthesis according to whether “h” was of maximum physiologically permitted amplitude or of minimum required amplitude.

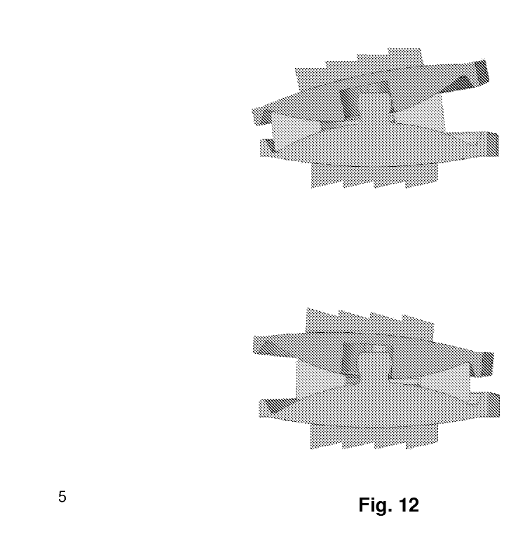

This limitation of transversal displacement will also be produced when the vertebrae connected by the prosthesis rotate. For example, in FIG. 12 can be seen the disposition of the different components of the prosthesis, in its semi-constrained configuration, when it is in the maximum flexed and extended positions. It can be seen likewise, the lateral displacement of the intermediate piece (3) with respect to the upper and lower pieces.

Finally, the advantages of the invention disc prosthesis are completely conclusive eliminating from the prosthesis, exclusively, the inconveniences of visco-elastic and above all from the constrained prosthesis that are continually forcing small articulations on not having a rotation axis with a variable position. Also it obviates the inconveniences of the non-constrained prosthesis such as the Charité and Moby-Disc enabling an exaggerated horizontal and uncontrolled displacement, a control which can be carried out by the invention prosthesis.

As previously indicated, the instantaneous axis of rotation in a relative movement between the two vertebrae of one vertebral unit or segment, is changing its position continually while a flexo-extension or the lateralization of the movement is being produced. This continuous change of the axis of rotation establishes a horizontal or transversal displacement of the vertebral body itself, which conditions that the prosthesis which is designed be fitted with these two possibilities, in such a way that this displacement may never be greater than the normal physiological displacement and therefore, that there must be a mechanism that limits the implant in this sense.

To achieve these two aspects, the invention prosthesis can be non-constrained, or well constrained or semi-constrained, so that it contains its instantaneous axis of rotation inside the intervertebral space itself, and that it enables limiting in some form this displacement that always goes to impact on the mechanical articulation of the later facets. At present with personal experience with constrained prostheses we have been able to observe some cases of facetary lumbar pain which reveals the tension to which said posterior articulations are submitted to with this kind of prosthesis.

In the invention prosthesis the existence of the central pivot (6) terminating in a small sphere is what really determines that the instantaneous axis of rotation remain included in the intervertebral space and on the other hand the play “h” due to the diameter of the orifice (8) of the upper piece (2) makes it possible to produce the continuous change of the instantaneous axis of rotation at the same time enabling a limited transversal displacement, since reaching the maximum degree of flexion or extension (approximately 12°), the intermediate piece (3), which is the biconcave disc with central orifice (7) of larger diameter than that of the central pivot (6), is displaced in one other sense at the same time as serving as a stop for limiting not only the transversal displacement but also flexion or extension.

Therefore, depending on the diameter of the hole (8) of the upper piece (2) we can obtain three different configurations.
when h=0 a constrained configuration is obtained;
if 0<h<1.5 mm, a semi-constrained configuration ; and
if 1.5<h<3 mm, a non-constrained configuration .

### 6.3.—Wear and Resistance of Material

Evidently all the constitutive elements of this implant have to be sufficiently resistant so they can support all the bio-mechanical requirements throughout the life of the subject that uses it. For this reason, the use of high wear resistant metal alloys of perfect bio-compatibility is imposed as an essential element in the same. Today, in this sense, there do not seem to be any big problems with the Chrome-Cobalt-Molybdenum, which has been selected for this invention prosthesis, which are more than proved in hip and knee prostheses.

Nevertheless, the same does not happen with the intermediate piece (3) which has to be made of a special material having a high degree of resistance and good tribological characteristics with the external pieces. For this, a polyethylene with a special reinforcing system which has a resistance three or four times higher than the resistance obtained with Ultra-High Molecular Weight Polyethylene. This is called Cross-Link Ultra-High Molecular Weight (CLUHMWPE). This material enables obtaining intermediate pieces in which an excellent polish can be obtained in order to have ideal sliding properties with the two convex semi-spheres (4, 5) of the pieces (1, 2), due to the very low friction coefficient, at the same time increasing the life of the implant and its resistance to wear.

On the other hand, the fact that the prosthesis has two sliding surfaces (4, 5) is important insofar as it enables obtaining better mobility and less wear with the two sliding surfaces than with a single one, since the friction is much less.

In addition, and as a very important aspect, said intermediate piece (3) is surrounded by a titanium metal ring (13). This ring confers to said intermediate piece, a substantially bigger mechanical resistance and enables its viewing by means of radiographies.

The prosthesis also incorporates a third articulation between the lower (1) and upper pieces (2) at the level of the sphere (6) of the central pivot and of the hole (8) of the upper piece (2). In this sense, this articulation works more as a pulley giving stability to the prosthesis as an element of the third sliding surface and in a certain way this stability can prevent any dislocation of the upper element of the same such as has already occurred with the Pro-Disc prosthesis to one of the authors and other surgeons.

### 6.4.—Approach

All the present prostheses are designed to be able to be implanted by purely anterior approach. However, there are significant number of patients that for anatomical reasons (complex situations of abdominal vessels) or in order to be intervened previously by way of the abdomen, are excluded as candidates of a disc prosthesis. Therefore, it is considered important to have the development of a disc prostheses that can be implanted from the side above all in the space L-4 L-5, that is the most complex due to its general coincidence with the bifurcation of the iliac veins and the difficulty of their displacement. This invention prosthesis complies with this requirement and depending on the arrangement of the exterior crests (11, 12) that both pieces (1) y (2) show, it is possible that this prosthesis be implanted by the lateral or anterior approach.

### 6.5.—Selection of Type of Implant

In this radical point, basically the best advantage of the invention prosthesis and, therefore, the possibility of success of the implant. Each patient will need a semi-constrained or constrained or non-constrained prosthesis, according to the type of degenerated discopathy it has, age, general muscularity and ligamental state, and the degree of instability of the segment to be treated.

If a Non-Constrained Discal Prosthesis is used foe a Hypernobile Degenerative Discopathy of a young man with good muscularity, segment mobility will be assured, but also will result that the stability remains guaranteed due to the strong muscularity that controls translation and rotary motion at all times.

On the contrary, if a Non-Constrained Discal Prosthesis is used in a Hypermobile Degenerative Discopathy for a young multipara woman with poor muscularity and great tegumentary laxitude, we will find that the vertebral segment will have all degrees of freedom for each anatomical plane, ensuring good mobility of the sector and continual changing of the instantaneous axis of rotation at every moment of movement, leaving the segment to the mercy of the state of elastic tension and indemnity of the muscular and capsule-ligamentary system, that as is presumed deficient, with which the stability of the segment may result in being very precarious and consequently will impact negative bio-mechanical overloads to the small articulations. In this case a Semi-Constrained or Constrained Prosthesis will have to be used.

Another disadvantageous situation that would occur in the case of Hypomobile Degenerative Discopathies in which in the case of applying a Constrained Prosthesis we would obtain a better stability but a practically nil mobility which really serves for nothing , since we would be facing a similar case of an intersomatic arthrodesis and that would lead us in time to a Degenerative Discopathy situation of the superadjacent disc or Transition Syndrome.

It is then, necessary to apply a type of prosthesis in each patient and use a Constrained, Non-Constrained or semi-Constrained model according to the mechanical conditions and anatomical state of conservation of small articulations of each segment and with a better or lesser degree of elasticity of the muscularity-ligamentary system, previous study with dynamic radiographies, TAC or computerized axial tomographs and Magnetic Resonance for each person.

### 7. INDICATION AND COUNTER INDICATIONS OF THE PROSTHESIS

The substitution of a degenerated disc with a disc prosthesis, apart from preserving the function of the changed segment, tries to protect the adjacent discs from the functional overload that represented the fixing of a segment of vertebral movement. Classically, the indications were established for the same situations in which the fusion was indicated, but with a large number of exceptions or reservations, to wit:
The bone must be of good quality, so that there must be no osteoporosis or other form of deficit in the receptor bone.
The discal space must keep a minimum height of 5 mm, to be able to admit the implant without great tension.
Almost all secondary situations of inflammation of the vertebrae or discs must be disposed of.
Also, there must be serious deformity or intervertebral displacement
There must be no associated discal hernias.
The way of entry must not show any previous intervention.
There must be no general serious illnesses or blood dyscrasias.
The patient must be of middle age between 40 and 60 years approximately.

At present, the indications have been amplified in a notable way above all in respect to the local situation and the number of segments to be treated. At present discal degeneration is accepted as an indication and polysegmentary post surgical instability. Also, the association of discal hernia does not constitute an absolute counter indication, as well as degenerative scoliosis deformity or moderate lysis or lystesis with the exception of the space L5-S1.

Summarizing, we can say that at present the prosthesis is indicated in the treatment of the following pathological discal situations:

### Absolute Indications:

Degenerative Discopathy, lumbar or cervical isolated monosegmental.
Degenerative Discopathy multisegmental lumbar or cervical
Adjacent disc syndrome secondary to vertebral fusion. Relative indications:
Degenerative Discopathy associated with multisegmental deformity
Post fractural syndromes with discal destruction and good bone reserve.
Regenerative Discopathies and instabilities associated with channel stenosis.
Single or multiple post-surgical discogenetic instabilities
Postdiscectomy Syndrome.
Secondary Discopathy vertebral inflammation with discal space and conserved bone reserve with sufficient interval clinical and analytical infection negativization.
Discopathy associated with non extruded hernia.
Situations of absolute indication associated with obesity, tobacco, diabetes or general or local illness that means increased risk.

Nevertheless absolute counter indications are maintained as general type, serious or tumoral illnesses, blood dyscrasias, pregnancies, etc., difficulties related to the approach method, on having been intervened previously for serious abdomen problems, etc. or unsuitable age of the good patient for excess or defect.

### 8. IMPLANTATION TECHNIQUE FOR THE PROSTHESIS

The said proper technique is started with the preparation of the disc, cleaning and removal of the cartilaginous plate of the vertebral plates until it reaches the fibrous discal ring in its whole lateral and rear perimeter.

Preparation of the disc, the distraction pins or rods in the previously determined points with a centering instrument and the distraction of the space are done with a set of distracter forceps and the help of expanding pincers so as not to damage the vertebral body.

Next, the height, width and lordosis of the prosthesis are determined with probes and the pitch of the grooves for the anchorage crests is cut for the prosthesis with double chisels.

Continuing and maintaining the final height of the implant by means of the distracter, the “packaged” prosthesis is introduced in the carrying pincers. The last step will be verifying by radiographies the correct position of the implant and closing the surgical incision.

The nature of the present invention having been sufficiently described, as well as a practical application of the same, it only remains to add that both its shape and materials and execution of the same, are susceptible to modifications, provided that they do not affect in any substantial way the characteristics that are claimed in the following.

1. An intervertebral disc prosthesis for universal application, of the type that is made up of three pieces:
one lower piece or plate (1) that is fixed to a lower vertebra, said piece having a lenticular shape in vertical section and a kidney in plan view with an upper surface (4) in the shape of a spherical helmet that ends in circular contention rim (14) and with a lower surface (9) also in the shape of a spherical helmet with less curvature than the curvature of the upper surface (4) and which ends in a flat edge;
an upper piece or plate (2) that is fixed to an upper vertebra, said piece having a lenticular shape in vertical section and of a kidney in plane view, with a lower surface (5) in the shape of a spherical helmet which terminates in a circular contention rim (14) and with an upper surface (10) also in the shape of a spherical helmet with less curvature than the curvature of the lower surface (5) y which terminates in a flat edge; and
an intermediate biconcave torroidal piece (3) that articulates in the other two lower (1) and upper pieces (2) by means of each of the upper and lower cavities of this intermediate piece (3) which are spherical with substantially the same curvature that said upper and lower surfaces (4, 5) of the lower (1) and upper pieces (2) respectively, with the object that these are in contact over the whole surface when these pieces (1, 2, 3) are mounted in their operative position. characterized because:
on the top of the spherical surface (4) of the lower piece (1) there is a pivot (6) with a spherical head arranged radially;
the upper piece (2) has a hole (8) of circular or elliptical shape
the intermediate piece (3) has a central hole (7) of circular shape and dimensions bigger than those of the hole (8) of the said upper piece (2); where, when the three pieces (1, 2, 3) that comprise the prosthesis are mounted in their operative position, said pivot with spherical head (6) penetrates the intermediate piece (3) through the central hole (7) and is housed in the hole (8) practically in the upper piece (2), said spherical head of said pivot (6) and said hole (8) forming a third articulation in the form of a pulley that provides stability to the disc prosthesis.

2. An intervertebral disc prosthesis according to claim 1, characterized because in said spherical head of said pivot (6) and said hole (8) there is a kind of play between the following:
without play, in that the diameter of the spherical head of said pivot (6) is substantially the same diameter of the hole (8) with which the prosthesis is a constrained prosthesis which enables only one degree of freedom in each anatomical plane and the position of the axis of rotation of a vertebra of a vertebral segment with respect to other vertebrae of said segment is forced by the design of the prosthesis;
with limited play in the range of approximately 0 to 1.5 mm, with which the prosthesis is a semi-constrained prosthesis, which enables more than a degree of freedom in each anatomical plane, which enables a slight transversal displacement of the vertebra with respect to the other vertebra of the same vertebral segment, and in that the position of said axis of rotation being determined not only by the prosthesis but also by the combined action of ligaments, articulating faces and muscles, and
with limited play in the range of approximately 1.5 to 3 mm, with which the prosthesis is a non-constrained prosthesis, which enables more than one degree of freedom in each anatomical plane, enabling a maximum transversal displacement, without exceeding the physiological limits, of one vertebra with respect to another vertebra in the same vertebral segment, of said axis of rotation and in the position of said axis of rotation being determined not only by the prosthesis but also by the combined action of ligaments, articular faces and muscles.

3. An intervertebral disc prosthesis according to claim 2, characterized because said play can be dimensioned with a value for the anterior-posterior direction of the vertebral segment and with another value for the lateral direction of the vertebral segment.

4. An intervertebral disc prosthesis according to claim 1, in which said pivot with spherical head (6) and the hole (8) are displaced in the middle saggital plane with respect to a central position in a way that the range of mobility of the prosthesis in flexion shall be more than in extension.

5. An intervertebral disc prosthesis according to claim 1, where said lower (1) and upper pieces (2) are formed by a highly wear resistant biocompatible metal alloy of chrome-cobalt-molybdenum, and in which the lower surfaces (9) of the lower piece (1) and upper (10) of said upper piece (2) is covered with a biocompatible material and easily osteo-integratable, cemented in a porous plasma of tantalum.

6. An intervertebral disc prosthesis according to claim 1, characterized because the said intermediate piece (3) is formed by a high degree of resistance and good tribological characteristics with the lower and upper pieces (1, 2) constituted of “cross link” polyethylene of ultra high molecular weight (CLUHMWPE) and said intermediate piece (3) is encircled by a titanium metal ring (13) which confers greater resistance and enables its viewing by radiography.

7. An intervertebral disc prosthesis according to claim 1, characterized because both said surfaces (9) of the lower piece (1) and (10) pf the upper piece (2) are provided in vertical sense with a pair of crests or spikes (11) and (12) respectively, parallel to each other, with two possible arrangements according to the selected way of entry:
anterior-posterior for implantation by anterior approach, and
lateral for its implantation by lateral approach.

8. An intervertebral disc prosthesis according to claim 1, where said circular contention edges (14) limit the displacement of said intermediate piece (3) and therefore the maximum degree of flexion, extension, and lateral flexion which said disc prosthesis enables.
